(19) United States
(12) Patent Application Publication
(10) Pub. No.: US 2022/0298518 A1
(43) Pub. Date: Sep. 22, 2022

(54) PLANT MODIFICATION METHOD USING AXILLARY BUD MERISTEM

(71) Applicant: KANEKA CORPORATION, Osaka-shi, Osaka (JP)

(72) Inventors: Haruyasu HAMADA, Hyogo (JP); Yozo NAGIRA, Hyogo (JP); Akira ENDO, Hyogo (JP); Naoaki TAOKA, Hyogo (JP)

(21) Appl. No.: 17/641,730

(22) PCT Filed: Sep. 2, 2020
(86) PCT No.: PCT/JP2020/033270

§ 371 Date: Mar. 9, 2022

(30) Foreign Application Priority Data
Sep. 10, 2019 (JP) 2019-164264

(51) Int. Cl. C12N 15/82

(52) U.S. Cl. CPC C12N 15/8206

(57) ABSTRACT

A method for transforming a plant includes exposing a shoot apex of an axillary bud of a plant body, and introducing, into the shoot apex, a microparticle coated with at least one kind of nucleic acid.

### TECHNCIAL FIELD

The present invention relates to an in planta transformation method for a plant using a shoot apex tissue.

### BACKGOUND

Currently widespread general methods of plant transformation include a method in which an exogenous gene is introduced into, for example, a protoplast, callus, or tissue piece of in vitro culture, with an electroporation method, via Agrobacterium turnefaciens, with a particle bombardment method, or any other methods. However, even if a gene is introduced into a cell or a tissue by such a method, it is difficult, in some plant species, to regenerate a plant body to produce a transformant due to the difficulty in tissue culture with, for example, a plant hormone. Also, the transformation efficiency is not sufficiently high and therefore a selective marker gene has to be introduced to perform a marker selection. Furthermore, a somatic mutation (somaclonal mutation) often occurs with the need of long-term tissue culture. Therefore, from the viewpoints of reduction in the efforts to produce a transformant of a plant and the safety of a transformant of a plant, there has been a demand for the development of an in planta transformation method and a method for producing an in planta transformed plant body without the involvement of tissue culture.

Meanwhile, for wheat, rice, and the like, transformation methods without calluses or tissue pieces of in vitro culture (in planta transformation methods) are also known. Known in planta transformation methods include a method in which a gene is directly introduced into an exposed shoot apex of an immature embryo or a fully mature embryo, or a young bud of a tuber with a particle bombardment method (NPL 1 and PTLs 1 and 2). Moreover, PTL 3 and NPL 2 disclose a method of infecting a fully mature embryo immediately after germination with Agrobacterium to introduce a gene.

Common in the above literatures, however, such an in planta transformation method for obtaining the next-generation seed using an immature embryo or a fully mature embryo after gene introduction is difficult to use for plants of vegetative propagation such as a potato or the like because many of the cultivated species thereof have the trait of male sterility. PTLs 1 and 2 use a young bud of a tuber, which is however low in survival rate after exposure of the shoot-apex meristem.

Moreover, PTLs 1 and 2 have not demonstrated that the transgene is transmitted to a next generation. Due to these factors, the above methods have not been widely used up to the present.

### CITATION LIST

### Patent Literature

PTL 1: International Publication No. WO2017/195905

PTL 2: International Publication No. WO2017/195906

PTL 3: International Publication No. WO2005/024034

### Non-Patent Literature

NPL 1: Bilang et al. (1993) Transient gene expression in vegetative shot apical meristems of wheat after ballistic microtargeting. Plant Journal (1993) 4, 735-744

NPL 2: Supartana et al. (2005) Development of simple and efficient in planta transformation for rice (Oryza sativa L.) using Agrobacterium tumefaciencs. Journal of Bioscience AND Bioengineering (2005) 4, 391-397

### SUMMARY OF INVENTION

### Technical Problem

The present invention provides a method for transforming a plant without calluses or tissue pieces of tissue culture in the presence of a plant hormone. The present invention also provides a method for transforming a plant without a plant hormone. The present invention further provides a method for transforming a plant without introducing a selective marker gene. The present invention further provides a highly reproducible method for producing a transformed plant.

### Solution to Problem

The present inventors conducted intensive studies for solving the above, and as a result have accomplished the present invention.

Specifically, the present invention includes the followings.

<1> A method for transforming a plant, the method comprising:
exposing a shoot apex of an axillary bud of a plant body; and
introducing, into the shoot apex, a microp article coated with at least one kind of nucleic acid.

<2> A method for producing a transformant of a plant, the method comprising:
exposing a shoot apex of an axillary bud of a plant body;
introducing, into the shoot apex, a microparticle coated with at least one kind of nucleic acid;
growing the shoot apex to obtain a plant body; and
selecting a transformed plant body from the plant body.

<3> A method for editing a plant genome, the method comprising:
exposing a shoot apex of an axillary bud of a plant body; and
introducing, into the shoot apex, a microparticle coated with at least one kind of nucleic acid and/or at least one kind of protein.

<4> A method for producing a genome-edited individual of a plant, the method comprising:
exposing a shoot apex of an axillary bud of a plant body;
introducing, into the shoot apex, a microparticle coated with at least one kind of nucleic acid and/or at least one kind of protein;
growing the shoot apex to obtain a plant body; and
selecting a genome-edited plant body from the plant body.

### Advantageous Effects of Invention

According to the present invention, it is possible to obtain a transformant of a plant with good reproducibility by using a shoot apex in an axillary bud as a target of transformation by a physical method, without tissue culture in the presence of a plant hormone, formation of calluses, and a selective marker gene.

### BRIEF DESCRIPTION OF DRAWINGS

FIG. 1 is a sketch of a potato, taken as an example, for describing each of the parts (Example 1).

FIG. 2A is a photograph that depicts transient expression of GFP in an axillary bud of a potato (Example 2; stolon).

FIG. 2B is a photograph that depicts transient expression of GFP in an axillary bud of a potato (Example 2; terrestrial lateral shoot).

FIG. 3 is a figure schematizing a method for obtaining a T₀-generation genome-edited individual, according to Example 2 in the present invention (Example 3).

FIG. 4 is a view that presents analysis results of introduction of a mutation into the target gene in a To plant individual (Example 4).

FIG. 5 is a view that presents analysis results of sequencing in a T₀ plant individual (Example 4).

### DESCRIPTION OF EMBODIMENTS

The present invention will be described below in detail.

An in planta transformation method according to the present invention includes a step of exposing a shoot apex of an axillary bud of a plant body, and a step of introducing, into the shoot apex, a microparticle coated with at least one kind of nucleic acid; and may further include other steps.

A method of the present invention for editing a plant genome includes a step of exposing a shoot apex of an axillary bud of a plant body, and a step of introducing, into the shoot apex, a microparticle coated with at least one kind of nucleic acid and/or at least one kind of protein; and may further include other steps.

The step of introducing the microparticle can introduce a microparticle coated with at least one kind of nucleic acid and/or at least one kind of protein so that deletion, insertion, or substitution of a base is introduced into a target site on the genome DNA of the plant. This makes it possible to obtain a plant body that is genome-edited by the introduction of deletion, insertion, or substitution of a base into the target site.

In the present invention, the term “stem” includes not only a natural stem obtained by cultivating (or culturing) a plant under natural conditions or conditions close to natural conditions, but also a stem obtained from a tissue culture such as an in vitro seedling or the like. As long as an artificial seed from which a transformable shoot apex can be obtained is developed, a stem formed therefrom can be used. The natural stem includes not only a stem obtained in an outdoor field or the like, but also a stem obtained through greenhouse cultivation. Also, it includes not only a stem obtained from a tuber but also a stem obtained from a seed. A stem obtained through direct reprogramming or the like can also be used as a stem in the present invention as long as a shoot apex can be obtained therefrom.

In the present invention, the term “stolon” refers to a kind of a lateral shoot generated from a subterranean node of the main stem. In the present invention, a newly generated stolon from a node of the stolon can also be used.

In the present invention, the term “axillary bud” refers to a tissue including a new bud shoot apex that is generated from the base of a leaf or the node in a stem or stolon.

It is preferable to use a tuber or a fully mature seed as a material for obtaining the axillary bud. The fully mature seed refers to what is fully matured as a seed in which a ripening process after pollination is completed.

The axillary bud is not particularly limited and may be appropriately selected depending on the intended purpose. Preferable is a terrestrial lateral shoot of a stem sprouting from a tuber or a subterranean lateral shoot (stolon) of a stem sprouting from a tuber. More preferable is a subterranean lateral shoot (stolon) of a stem sprouting from a tuber.

Particularly preferable is an axillary bud that is grown on a medium after a tuber is allowed to sprout.

In the present invention, the term “shoot apex” includes a growing point (shoot apical meristem) at the leading end of a stem, and a tissue including the growing point and several leaf primordia derived from the growing point. In the present invention, only a hemispherical (dome-shaped) growing point obtained by removing leaf primordia may be used as a shoot apex, or a shoot apex including a growing point and leaf primordia or a plant tissue including such a shoot apex may be used. A virus-free tissue is obtained by using only a growing point obtained by removing leaf primordia.

1. Step of Pre-Treatment for Transformation

The in planta transformation method according to the present invention can be applied to a wide variety of common seed-producing plants without being limited to plants of vegetative propagation. Therefore, plants to be subjected to the in planta transformation method according to the present invention are spermatophytes including angiosperms and gymnosperms. Angiosperms include monocotyledons and dicotyledons.

The type of monocotyledon is not limited, and examples thereof include plants of Gramineae, Liliaceae, Musaceae, Bromeliaceae, and Orchidaceae.

Examples of the plants of Gramineae include rice, wheat, barley, corn, oat, Japanese lawn grass, sorghum, rye, millet, and sugar cane. Examples of the plants of Liliaceae include Welsh onion and asparagus. Examples of the plants of Musaceae is banana. Examples of the plants of Bromeliaceae is pineapple. Examples of the plants of Orchidaceae include orchids.

Examples of the dicotyledons include plants of Brassicaceae, Leguminosae, Solanaceae, Cucurbitaceae, Convolvulaceae, Rosaceae, Moraceae, Malvaceae, Asteraceae, Amaranthaceae, and Polygonaceae.

Of these, plants of Solanaceae are preferable.

Examples of the plants of Brassicaceae include thale cress, Chinese cabbage, rape, cabbage, cauliflower, and Japanese radish. Examples of the plants of Legumineae include soybean, red mung bean, kidney bean, pea, black-eyed pea, and alfalfa. Examples of the plants of Solanaceae include tomato, eggplant, potato, tobacco, and red pepper. Of these, potato is preferable. Examples of the plants of Cucurbitaceae include Oriental melon, cucumber, cantaloupe melon, and watermelon. Examples of the plants of Convolvulaceae include morning glory, sweet potato (yam), and bindweed. Examples of the plants of Rosaceae include roses, strawberry, and apple. Examples of the plants of Moraceae include mulberry, fig, and rubber tree. Examples of the plants of Malvaceae include a cotton plant and kenaf. Examples of the plants of Asteraceae include lettuce and Stevia. Examples of the plants of Amaranthaceae include beet (sugar beet). Examples of the plants of Polygonaceae include buckwheat.

Examples of the gymnosperms include pine, Japanese cedar, ginkgo, and cycad.

In the in planta transformation method according to the present invention, first, a sterilized tuber is allowed to sprout. If necessary, light-exposure forced sprouting and light-exposure sprouting may be performed with a treatment of exposure to light. Sprouting is performed by cutting a tuber into sections and incubating them in a dark place in a growing medium or in vermiculite. A stolon is prepared by placing the node of a grown stem on an agar medium and incubating it in a dark place. When an in vitro seedling is produced, a sprouted bud or an axillary bud part is grown on an agar medium. The agar medium may be any medium that is typically used for culturing plants. It is preferable to use the MS medium, the Gamborg B5 Medium, the LS medium, or the like. More preferably, the MS medium can be used. For a potato, for example, the temperature for growing is, from 10 to 23° C. and more preferably 20° C.

2. Step of Exposing Shoot Apex by Removing Leaves and Leaf Primordia

Then, the internode of the stem of the above-prepared stolon or in vitro seedling is cut with a cutting tool such as a scalpel or the like. For a potato, a shoot apex is exposed by removing leaves and leaf primordia. The means for exposing can be any tool as long as it can remove leaves and leaf primordia under a stereoscopic microscope, and examples of the means include punching tools such as a needle with a diameter of about 0.2 mm, tweezers, pipettes, syringes, cutting tools, e.g. a scalpel and a cutter, or the like. Then, an excess portion of the stolon or the stem is removed with a cutting tool such as a scalpel or the like, and the stolon or lateral shoot, including the exposed shoot apex, is placed on an agar medium so that the shoot apex faces upward. In order to obtain a virus-free shoot apex, a scalpel may be replaced by a freshly sterilized scalpel at a final stage to isolate the shoot apex. In this case, a virus-free transformant can be obtained.

3. Step of Introducing Nucleic Acid and/or Protein Into Cells of Shoot Apex

A technique for introducing the intended gene is not particularly limited, and a known genetic engineering technique can be used. In general, a recombinant vector containing the intended gene is produced, and a nucleic acid (e.g., a recombinant vector), a protein, or the like can be introduced into a shoot apex of an axillary bud as a target using the Agrobacterium-mediated method, the electroporation method, the particle bombardment method, the PEG-calcium phosphate method, the liposome method, the microinjection method, the whisker method, the laser injection method, or the like.

The particle bombardment method is a method of bombarding a cellular tissue with a microparticle coated with a nucleic acid and/or a protein, and is effective in the case where Agrobacterium infection efficiency is low, such as the case of monocotyledons or the like.

A means of introducing the microparticle coated with a nucleic acid and/or a protein into the cells of a shoot apex is not particularly limited as long as the means can introduce the microparticle into plant cells. Examples of the means include a particle bombardment (gene gun) in the particle bombardment method.

A method of introducing the microparticle coated with a nucleic acid and/or a protein into the cells of a shoot apex is not particularly limited as long as the method can introduce a microparticle into plant cells. Examples of the method include a method of bombarding the microparticle with a particle bombardment (gene gun) in the particle bombardment method.

The microparticle is not particularly limited as long as it has high specific gravity to increase a penetration power into a cell, and it is chemically inert and thus less likely to harm a living organism. Examples of the microparticle include a metal microparticle and a ceramic microparticle. As the metal microparticle, a microparticle of a metal simple substance or an alloy microparticle may be used. Of the microparticle of a metal simple substance, a gold particle, a tungsten particle, and the like are particularly preferable.

Coating may be performed on the whole surface of the microparticle or on part of the surface of the microparticle.

A vector used in the present invention is not particularly limited, and examples thereof include pAL-based vectors (e.g., pAL51 and pAL156), pUC-based vectors (e.g., pUC18, pUC19, and pUC9), pBI-based vectors (e.g., pBI121, pBI101, pBI221, pBI2113, and pBI101.2), pPZP-based vectors, pSMA-based vectors, intermediate vectors (e.g., pLGV23 Neo and pNCAT), cauliflower mosaic virus (CaMV), bean common mosaic virus (BGMV), and tobacco mosaic virus (TMV).

A vector containing an intended gene can be produced, for example as described below. In order to insert an intended gene into a vector, a method can be used in which a purified DNA is cleaved with an appropriate restriction enzyme, inserted into a restriction enzyme site or multicloning site of an appropriate vector DNA, and ligated to the vector. An intended gene may be inserted into an intermediate vector through double cross-over. TA cloning, In-Fusion coning, and the like may also be used.

An intended gene is not particularly limited as long as the expression of the gene or the inhibition of the expression of the gene is desired. The intended gene may be an endogenous gene or an exogenous gene of a plant of interest. The exogenous gene may be derived from different species, and genes derived from, for example animals, plants, microorganisms, viruses, and the like can be used. Examples of such a gene include glycometabolism related genes, lipid metabolism related genes, useful substance (e.g., medicine, enzyme, pigment, and aroma component) production genes, plant growth controlling (promoting/inhibiting) genes, flowering regulation related genes, disease-and-pest resistance (e.g., insect damage resistance, nematode disease resistance, mold (fungus) disease resistance, bacterial disease resistance, and virus (disease) resistance) genes, environmental stress (e.g., low temperature, high temperature, dryness, salt, photoinhibition, and ultraviolet rays) resistance genes, transporter genes, flour milling properties related genes, baking properties related genes, noodle-making properties related genes, and site-specific nuclease genes. Other than a sense strand, the intended gene may also be introduced such that an antisense strand, ribozyme, RNAi, or the like is expressed, depending on the purpose of the gene introduction.

In the present specification, the term “genome editing” is a portion of the techniques called “new breeding techniques (NBT)”, and refers to disrupting a gene by cleaving a specific gene on the genome and introducing a mutation thereinto, or deleting, inserting, or substituting a DNA fragment in a site-specific manner, using a meganuclease, CRISPR-CAS, or the like. Using the genome editing technique makes it possible to disrupt a gene of interest with a high efficiency. With the gene disruption, only a gene of interest can be disrupted without traces of gene recombination, and therefore, such gene-disrupted plants are not treated as recombinant plants in some countries. Moreover, with the genome editing, site-specific insertion or substitution of a DNA fragment can be efficiently performed by linking the respective fragments that are homologous to the respective sequences of the two sides of a cleaved sequence to the two sides of a DNA fragment to be introduced into that site, respectively.

In the sense described above, the genome editing can be regarded as a technique different from a conventional plant transformation method, such as a direct introduction method or an Agrobacterium-mediated method, or the like, in which an exogenous gene is incorporated in a substantially random manner, and it may be thought that the genome editing technique is excluded from the definition of a transformation method. The genome editing technique has a feature of including a step of cleaving a genome DNA using a nuclease capable of targeting a cleavage site, or a nuclease with a guide RNA, and can be distinguished from a conventional transformation method without a nuclease capable of targeting, or a nuclease with a guide RNA. The term “using a nuclease, or a nuclease with a guide RNA” as used herein means that a nuclease protein may be introduced into a cell, and a DNA and/or RNA encoding a nuclease gene may be introduced into a cell to express a nuclease protein. Also, regarding the guide RNA, it is construed that an RNA may be introduced into a cell, and a DNA capable of expressing a guide RNA may be introduced to express a guide RNA.

Examples of proteins encoded by the site-specific nuclease genes include a zinc finger nuclease, a protein having zinc finger nuclease activity, and a TAL effector nuclease (TALEN). The zinc finger nuclease is a fusion protein of several zinc finger motifs that recognize a specific base and a FokI nuclease. The TALLEN is a fusion protein of a Transcription Activator Like (TAL) effector and a FokI nuclease. A site-specific nuclease includes another additional targeting technology, such as a meganuclease, RNA inducible CRISPR-Cas9, a leucine zipper, or the like.

Editing the genome by introducing a site-specific nuclease into a cell with the transformation method of the present invention, integrating it into a genome, and expressing it makes it possible to change or modify the expression of one or more gene products. Specifically, for a cell that contains and expresses a DNA molecule encoding one or more gene products, a CRISPR-Cas system which may contain a Cas protein and one or more guide RNAs targeting the DNA molecule is introduced into the cell, so that the one or more guide RNAs target genome gene loci of the DNA molecule encoding the one or more gene products, and the Cas protein cleaves the genome gene loci of the DNA molecule encoding the one or more gene products, thereby making it possible to change or modify the expression of the one or more gene products.

Cas protein and a guide RNA may be used in a naturally occurring manner (in combination), or may be used in combination that is not present in nature. In the present invention, the expression of two or more gene products may be changed or modified. The guide RNA may include a guide sequence fused to a tracr sequence.

The guide RNA has a length of at least 15, 16, 17, 18, 19, or 20 nucleotides, and the maximum number of nucleotides is preferably 30 or less, more preferably 25 or less, even more preferably 22 or less, and most preferably 20.

In preferable embodiments, cells to be transformed are plant cells. More preferably, they are cells of shoot apex meristem.

In the present invention, the Cas protein may contain one or more nuclear localization signals (NLSs). In some embodiments, the Cas protein is a type-II CRISPR enzyme. In some embodiments, the Cas protein is a Cas9 protein. In some embodiments, the Cas9 protein is Cas9 of Streptcoccus pneumoniae (S. pneumoniae), Streptcoccus pyogenes (S. pyogenes), or Streptcoccus thermophilus (S. thermophilus), and may also encompass mutant Cas9 derived from these organisms. The protein may be a Cas9 homolog or a Cas9 ortholog.

The Cas protein may be subjected to codon optimization for the expression in a eucaryotic cell. The Cas protein may direct the cleavage of one or two strands at a position where a target sequence is localized. In another aspect of the present invention, the expression of a gene product is reduced, and the gene product is a protein.

In addition to an intended gene, a promoter, an enhancer, an insulator, an intron, a terminator, a poly A addition signal, a selective marker gene, and the like can be ligated to the vector.

Two or more kinds of the intended gene may be inserted to one vector. Two or more kinds of the recombinant vector may be coated on one microparticle. For example, a recombinant vector containing the intended gene and a recombinant vector containing a drug-resistant vector may be prepared separately and mixed together, and the mixture may be coated on the microparticle to be bombarded into a plant tissue.

A promoter that is not derived from a plant may be used as long as a promoter is a DNA that can function in a plant body or a plant cell, and direct a constitutive expression or an expression in a specific tissue of a plant or at a specific growth stage of a plant. Specific examples thereof include a cauliflower mosaic virus (CaMV) 35S promoter, an El2-35S omega promoter, a promoter of nopaline synthase gene (Pnos), a ubiquitin promoter derived from corn, an actin promoter derived from rice, a PR protein promoter derived from tobacco, an ADH, and a RuBisco promoter. A sequence that enhances translational activity, for example an omega sequence of a tobacco mosaic virus, can be used to enhance the translation efficiency. Moreover, IRES (internal ribosomal entry site) can be inserted into a site on the 3′ downstream side of a promoter and the 5′ upstream side of a translation initiation codon as a translation initiation region so as to translate a protein from a plurality of coding regions.

The terminator may be any sequence as long as it is a sequence that can terminate the transcription of a gene transcribed by the above-mentioned promoter, and contains a poly A addition signal, and examples of the terminator include the terminator of a nopaline synthase (NOS) gene, the terminator of an octopine synthase (OCS) gene, and a CaMV 35S terminator.

Examples of the selective marker gene include herbicide resistance genes (e.g., a bialaphos resisitance gene, a glyphosate resistance gene (EPSPS), and a sulfonylurea resistance gene (ALS)), drug resistance genes (e.g., a tetracycline resistance gene, an ampicillin resistance gene, a kanamycin resistance gene, a hygromycin resistance gene, a spectinomycin resistance gene, a chloramphenicol resistance gene, a neomycin resistance gene, and the like), fluorescence or luminescence reporter genes (e.g., luciferase, ß-galactosidase, ß-glucuronidase (GUS), green fluorescent protein (GFP), and the like), and enzyme genes such as a neomycin phosphotransferase II (NPT II), dihydrofolate reductase, and the like. However, with the present invention, a transformant can be produced without introducing a selective marker gene.

The vector containing the intended gene is bombarded into the axillary bud of a potato with the particle bombardment method. The intended gene and/or protein can be coated on the surface of a microparticle (a microcarrier) and bombarded into plant cells with a gene gun. The microparticle for use is preferably a metal microparticle because it has high specific gravity to increase a penetration power into a cell, and it is chemically inert and thus less likely to harm a living organism. Of the metal microparticle, a gold particle, a tungsten particle, and the like are particularly preferably used.

In the particle bombardment method, an intended gene can be introduced into a plant cell as follows. First, a microparticle such as a gold particle, a. tungsten particle, or the like is washed and sterilized, and a nucleic acid (e.g., recombinant vector, linear DNA, RNA, or the like), CaCl₂, and spermidine are added to the microparticle while being stirred using a vortex mixer or the like so that the gold particle or the tungsten particle is coated (coating) with the DNA, and then the particle is washed with ethanol.

The particle diameter of the microp article for use is preferably 0.3 μm or more but 0.9 μm or less, more preferably 0.4 μm or more, even more preferably 0.5 μm or more, and particularly preferably 0.6 μm. The preferable upper limit of the particle diameter is 0.8 μm or less, more preferably 0.7 μm or less, and particularly preferably 0.6 μm.

The gold particles or tungsten particles are applied onto a macrocarrier film using PIPETMAN® (a pipette) or the like as uniformly as possible and then dried in a sterile environment such as in a clean bench or the like. The macrocarrier film and a plate on which a targeted shoot apex of an axillary bud is placed are mounted in a particle bombardment apparatus, and then a high-pressure helium gas is shot from a gas accelerating tube toward the macrocarrier film. The macrocarrier film stops at a stopping plate, but the gold particles pass through the stopping plate and enter the target placed below the stopping plate, so that the intended gene is introduced thereinto. When a microparticle coated with a protein is used, it is preferable to use a hydrophilic macrocarrier film.

The hydrophilic macrocarrier film may be formed by attaching a hydrophilic film to a macrocarrier film or applying hydrophilic coating onto a macrocarrier film. Examples of an approach for the hydrophilization of a film include approaches by use of a surfactant, a photocatalyst, and a hydrophilic polymer.

Examples of the hydrophilic polymer used in the above-mentioned approach include polymers of a hydrophilic monomer such as polyethylene glycol, hydroxyethyl methacrylate, hydroxypropyl methacrylate, dihydroxyethyl methacrylate, diethylene glycol methacrylate, triethylene glycol methacrylate, polyethylene glycol methacrylate, vinylpyrrolidone, acrylic acid, acrylamide, dimethylacrylamide, glucoxyoxyethyl methacrylate, 3-sulfopropylmethacryloxyethyldimethylammonium betaine, 2-methacryloyloxyethyl phosphorylcholine, 1-carboxydimethylmethacryloyloxyethyl methaneammonium, or the like.

Depending on the particle diameter of the microp article, the distance between the stopping plate and the targeted shoot apex is, for example preferably 9 cm or less, more preferably 8 cm or less, even more preferably 7 cm or less, and especially preferably 6 cm or less, and the minimum distance is, for example preferably 2 cm or more, more preferably 3 cm or more, and even more preferably 4 cm or more. Regarding the distance between the stopping plate and the target, an optimum value can be determined as appropriate through transient expression experiment or the like, depending on the type of microparticle, the particle diameter, gas pressure, and the like.

The gas pressure is for example, preferably 1,100 to 1,800 psi, and more preferably 1,300 to 1,500 psi, depending on the type of microparticle and the distance to the target. Regarding the gas pressure, an optimum value can be determined as appropriate through transient expression experiment or the like, depending on the type of microparticle, the type of target, the distance between the target and the stopping plate, and the like.

In the transformation method according to the present invention, the number of shots for bombarding a shoot apex with the microparticle is preferably two shots or more, more preferably three shots or more, and even more preferably four shots or more. The upper limit of the number of shots for bombarding a shoot apex with the microparticle is preferably twenty shots or less, more preferably fifteen shots or less, and even more preferably ten shots or less. Regarding the number of shots for bombardments, an optimum number is determined as appropriate through transient expression experiment or the like.

In the cell bombarded with the microparticle, the nucleic acid is released from the microparticle and is integrated with a genome DNA, and thus a transformed cell is obtained. However, when a nucleic acid such as geminivirus that is proliferated in a plasmid shape or an artificial chromosome is introduced, a cell may be transformed without the integration. Also, with the transformation method according to the present invention, an exogenous gene can be introduced into an organelle. In such a case, it is preferable to use a gene to which a promoter that is expressed specifically in an organelle is operably linked.

4. Other Steps

Examples of the above other steps include a step of growing the shoot apex bombarded with the microparticle to obtain plant bodies, and a step of selecting an intended plant body from the plant bodies.

The step of growing the shoot apex bombarded with the particles to obtain a plant body is not particularly limited and may be appropriately selected depending on the intended purpose. This step is performed by, for example, a method in which the shoot apex of the axillary bud transformed is grown on an agar medium for about one month and then transferred to soil.

A bombarded shoot apex can be grown on a normal medium without applying selective pressure using a drug or the like (i.e., on a medium free from antibiotics or the like) to obtain a transformant, and a drug resistance gene may be further introduced. When a drug resistance gene is introduced, a drug can be used to selectively culture transformed cells. For example, a sulfonylurea-based herbicide, chlorsulfuron (the resistance against this herbicide can be acquired by introducing a mutated ALS gene (acetobutyrate synthase gene)), or the like is known as a selection drug suitable for shoot apex culture.

When a drug resistance gene is introduced, the drug resistance gene and the intended gene may be present in the same vector or separate vectors. When the drug resistance gene and the intended gene are inserted into separate vectors, and are integrated into separate chromosomes, there is an advantage that self-pollination or backcross is performed to produce progenies so that an intended gene-introduced plant individual and a drug resistance gene-carrying plant individual can be separately obtained.

The method of the present invention for producing a transformant of a plant includes a step of exposing a shoot apex of an axillary bud of a plant body, a step of bombarding, into the shoot apex, a microparticle coated with at least one kind of nucleic acid, a step of growing the shoot apex to obtain a plant body, and a step of selecting a transformed plant body from the plant body.

The method of the present invention for producing a genome-edited individual of a plant includes a step of exposing a shoot apex of an axillary bud of a plant body, bombarding, into the shoot apex, a microparticle coated with at least one kind of nucleic acid and/or at least one kind of protein, a step of growing the shoot apex to obtain a plant body, and selecting a genome-edited plant body from the plant body.

Each of the above steps is as described above.

With the above method, an intended gene-introduced plant body or a genome-edited plant body can be produced. In the thus-produced plant, the intended gene is stably expressed or the expression of the intended gene is suppressed, which is normally inherited (transmitted) to progenies.

The gene introduction efficiency or the genome editing efficiency into a potato can be evaluated as follows.

For the gene introduction efficiency, by extracting DNA from a grown individual that has been subjected to a gene introduction process and performing PCR and/or Southern blotting, it can be detected whether the intended gene has been genome-edited. The genome editing efficiency is calculated from the number of explants used for the gene introduction and the number of grown individuals carrying an exogenous gene.

For the genome editing efficiency of the intended gene, by extracting DNA from a grown individual that has been subjected to a gene introduction process and treating a PCR product with a restriction enzyme, it can be detected whether the intended gene has been genome-edited. The genome editing efficiency is calculated from the number of explants used for the gene introduction and the number of grown individuals carrying a target mutation. For the grown individuals confirmed to carry the target mutation, the presence or absence of RNA expressed from the intended gene is evaluated. The presence or absence of RNA can be confirmed, for example by the RT-PCR method. It may be detected through Northern blotting.

The presence or absence of a protein expressed from the genome-edited target gene can be evaluated. The presence or absence of a protein can be confirmed through staining of plant section, electrophoresis, ELISA, RIA, dot-immunobinding assay, and/or Western blotting. The genome editing efficiency of the intended gene is calculated from the number of explants used for the gene introduction and the number of grown individuals in which the presence (or absence) of a protein reflecting genome editing of the intended gene has been confirmed.

### EXAMPLES

The present invention will be described below in detail by way of Examples but should not be construed as being limited to these Examples in any way.

### Example 1 Study of an Optimal Tissue for Transformation

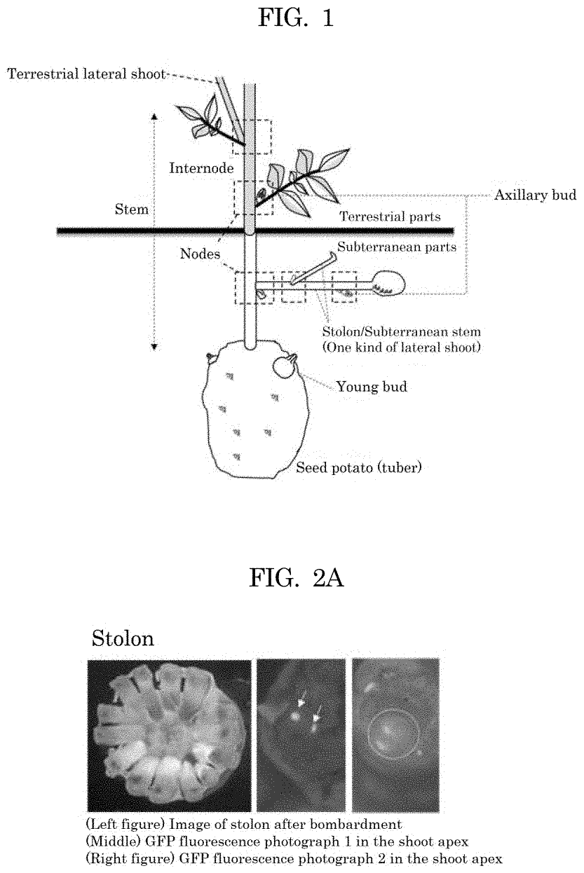

For transformation, leaf primordia are removed to expose a shoot apex. In order to select an optimal tissue for transformation, the shoot apexes of various tissues were exposed (FIG. 1), followed by calculating the survival rates thereafter.

1. Preparation of Tissue

(1) Preparation of Young Bud

A tuber of a potato (variety: Danshakuimo) was instantly immersed in ethanol (99.5%) and placed on KIMTOWEL (a trade name; a disposable wipe), followed by incubating at 22° C. After sprouting, a base part of a sprouted bud with respect to the tuber was cut with a sterile knife to separate a young bud from the tuber. Under a stereoscopic microscope, the leaf primordia were removed with a leading end of a needle (diameter: 0.20 mm). This was placed on a MS-sucrose medium (4.3 g/L MS salt, MS vitamin, 30 g/L sucrose, 0.98 g/L MES, 3% PPM (plant preservative mixture, NACALAI TESQUE, INC.), 7.0 g/L phytagel, pH 5.8) so that the shoot apex would face upward.

(2) Preparation of Subterranean Lateral Shoot

A tuber of a potato (variety: Russet Burbank) was instantly immersed in ethanol (99.5%) and placed on KIMTOWEL (a trade name; a disposable wipe), followed by incubating at 22° C. After sprouting, the tuber was cut into half with a sterile knife and transplanted into a disposable plant cell culture container containing the MS-sucrose medium, followed by growing for about one month in a long day condition (22° C., day length of 16 hours). The internode of a stem of a regenerated plant individual was cut with a sterile knife. The node was transplanted again into a disposable plant cell culture container containing a MS-sucrose medium, followed by growing under the same conditions. The stem of a plant body regenerated after this procedure had been repeated twice or more was used for an experiment. The internode and the leaves at the node were cut with a sterile knife. Under a stereoscopic microscope, the leaf primordia were removed with a leading end of a needle (diameter: 0.20 mm). This was placed on the MS-sucrose medium so that the shoot apex would face upward.

The same procedure as described above was performed also when preparing a subterranean lateral shoot using a potato (variety: Danshakuimo).

(3) Preparation of Stolon

A tuber of a potato (variety: Danshakuimo) was instantly immersed in ethanol (99.5%) and placed on KIMTOWEL (a trade name; a disposable wipe), followed by incubating at 22° C. After sprouting, the tuber was cut into half with a sterile knife and transplanted into a pot containing a seedling raising medium for horticulture. After that, in a dark place and in a growth chamber set to 22° C., it was grown until a stem elongated. The internode was cut with a sterile knife. The node was transplanted into a disposable plant cell culture container containing the MS-sucrose medium, followed by growing until a stolon elongated in a dark place and in a growth chamber set to 22° C. The internode of the stolon was cut with a sterile knife. Under a stereoscopic microscope, the leaf primordia were removed with leading end of a needle (diameter: 0.20 mm). This was placed on the MS-sucrose medium so that the shoot apex would face upward.

2. Calculation of Survival Rate

The plates prepared in Examples 1-(1) and (2) were grown for about three weeks in a long day condition (22° C., day length of 16 hours). The plate prepared in Example 1-(3) was similarly grown in a dark place at 22° C. for about three weeks. Regarding an individual with an elongated stem or stolon as a living individual, the survival rate of each tissue after the exposure of the shoot apex was calculated (Table 1). The survival rate of the young bud was low; i.e., 16%, while those of the subterranean lateral shoot and the stolon were high; i.e., 100% and 90%. The survival rate after the exposure of the shoot apex was found to be greatly different depending on the tissue.

TABLE 1

| | Number of | | Survival rate after |
|---|---|---|---|
| | individuals | Living | the exposure of the |
| Tissue | used | individuals | shoot apex |
| Young bud | 25 | 4 | 16% |
| Terrestrial | 20 | 20 | 100% |
| axillary bud | | | |
| (Russet Burbank) | | | |
| Terrestrial | 20 | 18 | 90% |
| axillary bud | | | |
| (Danshakuimo) | | | |
| Stolon | 20 | 18 | 90% |

### Example 2 Obtainment of Transformant

When the axillary bud (terrestrial lateral shoot or stolon) was used, the survival rate after the exposure of the shoot apex was found to be high. Using this tissue makes it possible to obtain a transformant with the in planta method.

1. Preparation of Tissue

A procedure follows the method described in Examples 1-(2) and (3). Each tissue was placed on the MS-sucrose medium at 15 to 20/plate so that the shoot apex would face upward.

2. Gene Introduction with the Particle Bombardment Method

Gene introduction to the shoot apex of a potato was performed with the particle bombardment method in the following manner.

(1) Preparation and Bombardment of Gold Particles

30 mg of gold particles of 0.6 μm was weighed. 500 μL of 70% ethanol was added to the gold particles, followed by suspending well with a vortex. After that, the gold particles were precipitated through centrifugation, and the ethanol was removed. The gold particles were washed three times with NUCLEASE-FREE WATER (a trade name; nuclease free water) (QIAGEN K.K.), followed by addition of 500 μL of NUCLEASE-FREE WATER (a trade name; nuclease free water) (QIAGEN K.K.), to prepare a gold particles stock solution.

In the Examples, as a transgene, plasmid DNA (pUC-based plasmid) containing a fluorescence reporter gene GFP (S65T) was used. This gene is designed to express under control of a 35S promoter derived from a Cauliflower mosaic virus. As a terminator, a terminator of a nopaline synthase (NOS) gene is added.

A plasmid DNA solution (1 μg/μL) purified using QIAGEN® PLASMID MIDI KIT (a trade name; a kit for plasmid preparation) (QIAGEN K.K.) was placed into a 1.5-mL tube at 5 μg per 750 μg of the gold particles. Before use, the sterile gold particle-containing solution was thoroughly suspended using an ultrasonic generator (ultrasonic washer UW-25 manufactured by Taga Electric Co., Ltd.), and placed into the above-mentioned tube in an appropriate amount and stirred with pipetting. Next, 25 μL of 2.5 M CaCl₂ (NACALAI TESQUE, INC.) and 10 μL of 0.1 M Spermidine (NACALAI TESQUE, INC.) per 750 μg of the gold particles were added to the above-mentioned tube. Immediately after mixing, the resultant mixture was vigorously suspended for 5 minutes using a vortex mixer. The mixture was left to stand at room temperature for 10 minutes, and was then centrifuged at 9,100×g for 2 seconds. The supernatant was removed, and the precipitation was washed with 70% ethanol and then 99.5% ethanol. Lastly, the supernatant was removed, and 24 μL of 99.5% ethanol was added thereto and suspended well. In a clean bench, 6 μL of the suspension was poured to the center of a macrocarrier, and the macrocarrier was then air-dried.

The particle bombardment was performed with BIOLISTIC PDS-1000/HE PARTICLE DELIVERY SYSTEM (a trade name; a system introducing microcarriers into cells) (BIO-RAD Laboratories, Inc.). Bombardment pressure was set to about 94.9 kgf/cm² (1,350 psi), and the distance from a stopping screen to a target tissue was set to 3.5 cm. The samples were bombarded with the particles at 4 shots per dish. After bombardment, the samples were left to stand overnight in a dark place at 22° C.

(2) Confirmation of Expression of GFP Protein

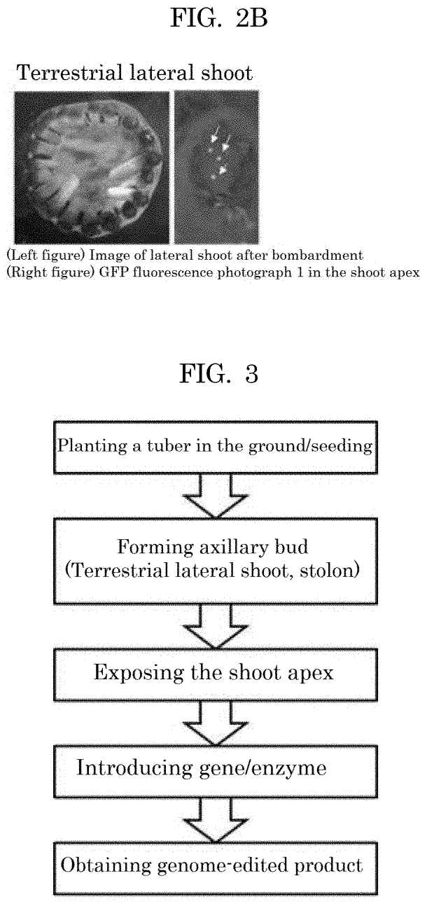

GFP fluorescence (excitation: 470/40, absorption: 525/50) in the shoot apex of the axillary bud was observed under a stereoscopic fluorescence microscope (MZFL III (a trade name; a stereoscopic fluorescence microscope) manufactured by Leica Microsystems). As a result, GFP fluorescence was observed in spots or over the entire shoot apex in the shoot apex of the stolon (FIG. 2A) and the shoot apex of the terrestrial lateral shoot (FIG. 2B). This results indicates that gene introduction into the shoot apex of the axillary bud is possible with the particle bombardment.

3. Obtainment of Transformant

The individuals that had been confirmed for GFP fluorescence in the shoot apex of the axillary bud of the terrestrial lateral shoot were transplanted into a disposable plant cell culture container containing the MS-sucrose medium, followed by growing for about two weeks in a long day condition (22° C., day length of 16 hours). Whether an exogenous gene was introduced was investigated in a leaf differentiated from the shoot apex. Specifically, genomic DNA was extracted from a grown leaf (50 mg) with the benzyl chloride method, and PCR was performed using the extracted genomic DNA as a template and the primers that had been produced based on a sequence specific to the 35S promotor.
35S-F: CCAGAGGGCTATTGAGACTTTTC (SEQ ID NO: 1)
35S-R: ATATAGAGGAAGGGTCTTGCGAA (SEQ ID NO: 2)

A PCR reaction mixture was prepared by mixing the genomic DNA (1.0 μL), KOD FX NEO (a trade name; a PCR enzyme) (TOYOBO CO., LTD.) 0.4 U, accompanying 2× buffer (5 μL), 0.4 mM dNTPs, and the pair of primers (0.2 μM each) with sterile distilled water such that the total volume was 10 μL.

PCR was performed using TAKARA PCR THERMAL CYCLER DICE (a trade name; a PCR device) (TAKARA BIO INC.) at 32 cycles of reaction of 98° C. for 10 seconds and 68° C. for 1 minute. After the PCR reaction, each PCR product (5 μL) was subjected to 1.0% agarose gel electrophoresis and stained with ethidium bromide.

As presented in Table 2, 16 individuals out of 236 individuals that were subjected to the gene introduction treatment, a signal indicative of insertion of the exogenous gene was detected, and thus the transformation efficiency was 6.8%. As can be seen from the above, using the present method makes it possible to efficiently obtain a transformant.

TABLE 2

| | Number of | Number of | |
|---|---|---|---|
| | individuals | individuals with | |
| | treated | GFP expression in | |
| Tested | for gene | the shoot apex of | Number of |
| tissue | introduction | the axillary bud | transformants |
| Terrestrial | 236 | 168 | 16 (6.8%) |
| axillary bud | | | |
| (Danshakuimo) | | | |

### Example 3 Obtainment of Genome-Edited Individual

When the axillary bud (terrestrial lateral shoot or stolon) was used, the survival rate after the exposure of the shoot apex was found to be high. Using this tissue makes it possible to obtain a transformant with the in planta method (FIG. 3).

1. Preparation of Tissue

A procedure follows the method described in Examples 1-(2) and (3). Each tissue was placed on the MS-sucrose medium at 15 to 20/plate so that the shoot apex would face upward.

2. Protein Introduction with the particle bombardment method

Protein introduction to the shoot apex of a potato was performed with the particle bombardment method in the following manner.

(1) Preparation and Bombardment of Gold Particles

60 mg of gold particles of 0.6 μm was weighed. 500 μL of 70% ethanol was added to the gold particles, followed by suspending well with a vortex. After that, the gold particles were precipitated through centrifugation, and the ethanol was removed. The gold particles were washed three times with NUCLEASE-FREE WATER (a trade name; nuclease free water) (QIAGEN K.K.), followed by addition of 500 μL of NUCLEASE-FREE WATER (a trade name; nuclease free water) (QIAGEN K.K.) to prepare a gold particles stock solution.

In the Examples, a mixture (1 μg/μL) of crRNA and tracrRNA (FASMAC CO., LTD.) was used as gRNA. A Cas9 protein used is, for example, Streptococcus pyogenes-derived Cas9 (TAKARA BIO INC.).

crRNA-GBSS1-tg1:
(SEQ ID NO: 3)
AGGGCUGUUAACAAGCUUGAguuuuagagcuaugcuguuuug
crRNA-GBSS1-tg2:
(SEQ ID NO: 4)
GGGCUGUUAACAAGCUUGAUguuuuagagcuaugcuguuuug
crRNA-GBSS1-tg3:
(SEQ ID NO: 5)
UACUAAGGUAACACCCAAGAguuuuagagcuaugcuguuuug
crRNA-ALS1/2:
(SEQ ID NO: 6)
CAAGUGCCGAGGAGGAUGAUguuuuagagcuaugcuguuuug
tracrRNA:
(SEQ ID NO: 7)
AAACAGCAUAGCAAGUUAAAAUAAGGCUAGUCCGUUAUCAACUUGAAAAA
GUGGCACCGAGUCGGUGCU

Each of the above gRNA or the gRNA mixtures (crRNA-GBSS1-tgl, crRNA-GBSS1-tg2, crRNA-GBSS1-tg3) (5 μg), the Cas9 protein (12 μg), 10× CUTSMART® buffer (a buffer) (5 μL), and RIBOLOCK RNASE INHIBITOR (a trade name; a RNase inhibitor) (1.0 μL) were added, and the mixture was filled up to a total of 20 μL with sterile distilled water. The mixture was left to stand still at room temperature for 15 minutes. Then, gold particles (1500 μg) were added thereto, and the mixture was left to stand still on ice for 10 minutes. Discarding the supernatant was followed by addition of NUCLEASE-FREE WATER (a trade name; nuclease free water) (QIAGEN K.K.) (26 μL). The resultant is used as a gold particle/Cas9 complex. In a clean bench, a hydrophilic film (3M Company, SH2CLHF) cut into from 1.0 to 1.5 cm square was attached to the center of a macrocarrier. To this, the gold particle/Cas9 complex (6 μL) was poured, followed by air drying. The diameter of the gold particles was 0.6 μm. The amount of the gold particles was adjusted so that it would be 375 μg per shot of bombardment, and four shots of bombardment were performed per plate.

(2) Growth of Individuals Treated for Protein Introduction

The individuals treated for protein introduction were left to stand still overnight and then transplanted into a disposable plant cell culture container (Sigma-Aldrich Japan K.K.) containing the MS-sucrose medium, followed by growing for from three to four weeks in a long day condition (22° C., day length of 16 hours).

(3) Analysis of Introduction of Mutation into Target Gene in T₀ Plant Individual

The obtained plant body is investigated for whether a mutation is introduced into a target gene (GBSS1 gene or ALS1/2gene). Specifically, genomic DNA was extracted from the grown entire plant body (50 mg) with the benzyl chloride method, and PCR was performed using the extracted genomic DNA as a template and the primers that had been produced based on a sequence specific to each gene.

GBSS1-F:
(SEQ ID NO: 8)
CTTGCCTACTGTAATCGGTGATAA
GBSS1-R:
(SEQ ID NO: 9)
TTTGACCTGCAGATAAAGTAGCG
ALS1/2-F:
(SEQ ID NO: 10)
GGTTCCCTGGTGTTTGCATT
ALS1/2-R:
(SEQ ID NO: 11)
GCTTCACGAACAACCCTAGG

A PCR reaction mixture is prepared by mixing the genomic DNA (1.0 μL), KOD FX NEO (a trade name; a PCR enzyme) (TOYOBO CO., LTD.) 0.4 U, accompanying 2× buffer (5 μL), 0.4 mM dNTPs, and the pair of primers (0.2 μM each) with sterile distilled water such that the total volume is 10 μL.

PCR was performed using TAKARA PCR THERMAL CYCLER DICE (a trade name; a PCR device) (TAKARA BIO INC.) at 35 cycles of reaction of 98° C. for 10 seconds and 68° C. for 1 minute. After the PCR reaction, each PCR product (1 μL), accompanying 10× buffer (1 μL), and an appropriate restriction enzyme (5 U) are added, and the mixture is filled up to a total of 10 μL with sterile distilled water. After the mixture has been allowed to react at 37° C. for 3 hours, the resultant mixture is subjected to 1.0% agarose gel electrophoresis and stained with ethidium bromide.

The PCR products were completely cleaved by the restriction enzyme in the wild-type strain, whereas remaining uncut portion was generated from the PCR products in the mutated individual. DNA can be extracted from a band of the uncut product, followed by purifying and then sequencing, to determine the individual having the mutation introduced into the target gene sequence, as the target gene mutation-introduced individual. Out of the individuals treated for protein introduction, the introduction of a mutation into the target gene can be confirmed in T₀-generation plant bodies at a certain proportion.

### Example 4 Obtainment of Genome-Edited Individual

Whether the axillary bud (terrestrial lateral shoot) can be used to obtain a genome-edited individual with the in planta method was investigated.

1. Preparation of Tissue

A procedure follows the method described in Example 1-(2). Each tissue was placed on the MS-sucrose medium at 15 to 20/plate so that the shoot apex would face upward. The varieties used were Russet Burbank and May queen.

2. Protein Introduction with the Particle Bombardment Method

Protein introduction to the shoot apex of a potato was performed with the particle bombardment method in the following manner.

(1) Preparation and Bombardment of Gold Particles

180 mg of gold particles of 0.6 μm was weighed. 500 μL of 70% ethanol was added to the gold particles, followed by suspending well with a vortex. After that, the gold particles were precipitated through centrifugation, and the ethanol was removed. The gold particles were washed three times with NUCLEASE-FREE WATER (a trade name; nuclease free water) (QIAGEN K.K.), followed by addition of 500 μL of NUCLEASE-FREE WATER (a trade name; nuclease free water) (QIAGEN K.K.), to prepare a gold particles stock solution.

A mixture (1 μg/μL) of crRNA and tracrRNA (FASMAC Co., Ltd.) was used as gRNA. A Cas9 protein used is Streptococcus pyogenes-derived Cas9 (New England BioLabs Inc.).

crRNA-PDS-tg1:
(SEQ ID NO: 12)
GGACUCUUGCCAGCAAUGCUguuuuagagcuaugcuguuuug
tracrRNA:
(SEQ ID NO: 7)
AAACAGCAUAGCAAGUUAAAAUAAGGCUAGUCCGUUAUCAACUUGAAAAA
GUGGCACCGAGUCGGUGCU

Each of the crRNA-PDS-tgl (2.5 μg), tracrRNA (2.5 μg), the Cas9 protein (16 μg), 10×Cut Smart buffer (4 μL), and RIBOLOCK RNASE INHIBITOR (a trade name; a RNase inhibitor) (1.0 μL) were added, and the mixture was filled up to a total of 20 μL with sterile distilled water. The mixture was left to stand still at room temperature for 10 minutes. Then, 5 μL of 1,4-bis (3-oleoylamidopropyl) piperazine/histone H1 protein mixture (3:1, 1330 μg/mL) was added thereto and gold particles (2250 μg) were added thereto, and the mixture was left to stand still on ice for 10 minutes. Discarding the supernatant was followed by addition of NUCLEASE-FREE WATER (a trade name; nuclease free water) (QIAGEN K.K.) (26 μL). The resultant was used as a gold particle/Cas9 complex. In a clean bench, a hydrophilic film (3M Company, SH2CLHF) cut into from 1.0 to 1.5 cm square was attached to the center of a macrocarrier. To this, the gold particle/Cas9 complex (6 μL) was poured, followed by air drying. The diameter of the gold particles was 0.6 μm. The amount of the gold particles was adjusted so that it would be 562.5 μg per shot of bombardment, the gas pressure was adjusted to from 1750 to 1800 psi, and two shots of bombardment were performed per plate.

(2) Growth of Individuals Treated for Protein Introduction

The individuals treated for protein introduction were left to stand still overnight and then transplanted into a disposable plant cell culture container (Sigma-Aldrich Japan K.K.) containing the MS-sucrose medium, followed by growing for from three to four weeks in a long day condition (22° C., day length of 16 hours).

(3) Analysis of Introduction of Mutation into Target Gene in T₀ Plant Individual

The obtained plant body was investigated for whether a mutation was introduced into a target gene (PDS gene). Specifically, genomic DNA was extracted from the grown entire plant body (50 mg) with the benzyl chloride method, and PCR was performed using the extracted genomic DNA as a template and the primers that had been produced based on a sequence specific to each gene.

PDS-F:
(SEQ ID NO: 13)
TTTCCCCGAAGCTTTACCCG
PDS-R:
(SEQ ID NO: 14)
ATCTGTCACCCTATCCGGCA

A PCR reaction mixture was prepared by mixing the genomic DNA (1.0 μL), KOD FX NEO (a trade name; a PCR enzyme) (TOYOBO CO., LTD.) 0.4 U, accompanying 2× buffer (5 μL), 0.4 mM dNTPs, and the pair of primers (0.2 μM each) with sterile distilled water such that the total volume was 10 μL.

PCR was performed using TAKARA PCR THERMAL CYCLER DICE (a trade name; a PCR device) (TAKARA BIO INC.) at 35 cycles of reaction of 98° C. for 10 seconds and 68° C. for 1 minute. After the PCR reaction, each PCR product (1 μL), accompanying 10× buffer (1 μL), and an appropriate restriction enzyme (BsrDI) (5 U) were added, and the mixture was filled up to a total of 10 μL with sterile distilled water. After the mixture had been allowed to react at 37° C. for 3 hours, the resultant mixture was subjected to 1.0% agarose gel electrophoresis and stained with ethidium bromide.

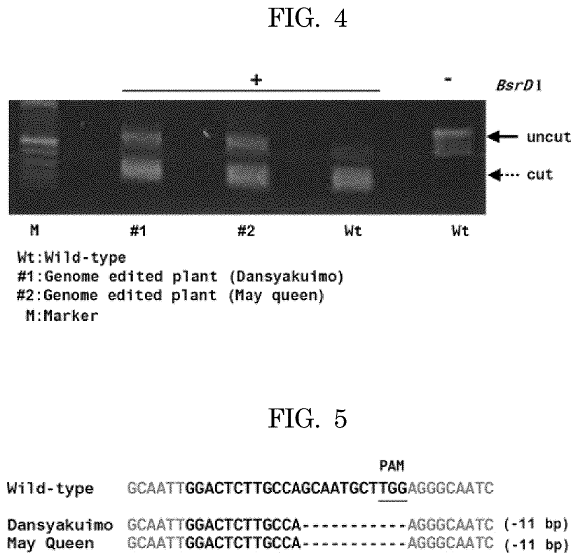

The PCR products were completely cleaved by the restriction enzyme in the wild-type strain, whereas remaining uncut portion was generated from the PCR products in the mutated individual (FIG. 4).

In FIG. 4, Lane 1 is a size marker (M), Lane 2 is the genome-edited individual of Danshakuimo (#1), Lane 3 is the genome-edited individual of May queen (#2), Lane 4 is the wild-type strain (Wt), and Lane 5 is the wild-type strain (Wt) without a restriction enzyme.

DNA was extracted from a band of the uncut product, followed by purifying and then sequencing. As a result, it was found that a mutation of 11-base deletion was introduced near the target gene sequence (FIG. 5). The genome-edited individual was obtained at a proportion of one out of 69 individuals (1.4%) for Danshakuimo and at a proportion of one of 78 individuals (1.3%) for May queen. Out of the individuals treated for protein introduction, the introduction of a mutation into the target gene were able to be confirmed in To-generation plant bodies at a certain proportion.

Also for other plants of Solanaceae, a transformant and a genome-edited individual can be obtained with substantially the same method as for a potato.

Aspects of the present invention are, for example as follows.
<1> A method for transforming a plant, the method comprising:
exposing a shoot apex of an axillary bud of a plant body; and
introducing, into the shoot apex, a microparticle coated with at least one kind of nucleic acid.

<2> The method according to <1>above, wherein the axillary bud of the plant body is an axillary bud of a plant body grown on a medium.

<3> The method according to <1>or <2>above, wherein the axillary bud is an axillary bud of a lateral shoot.

<4> The method according to <3> above, wherein the lateral shoot is a stolon.

<5> The method according to any one of <1> to <4> above, wherein the plant is any one selected from the group consisting of plants of Solanaceae.

<6> The method according to <5> above, wherein the plants of Solanaceae are potatoes.

<7> A method for producing a transformant of a plant, the method comprising:
exposing a shoot apex of an axillary bud of a plant body;
introducing, into the shoot apex, a microparticle coated with at least one kind of nucleic acid;
growing the shoot apex to obtain a plant body; and
selecting a transformed plant body from the plant body.

<8> A method for editing a plant genome, the method comprising:
exposing a shoot apex of an axillary bud of a plant body; and
introducing, into the shoot apex, a microparticle coated with at least one kind of nucleic acid and/or at least one kind of protein.

<9> The method according to <8> above, wherein the axillary bud of the plant body is an axillary bud of a plant body grown on a medium.

<10> The method according to <8> or <9> above, wherein the axillary bud is an axillary bud of a lateral shoot. <11> The method according to <10> above, wherein the lateral shoot is a stolon.

<12> The method according to any one of <8> to <11> above, wherein the plant is any one selected from the group consisting of plants of Solanaceae.

<13> The method according to <12> above, wherein the plants of Solanaceae are potatoes.

<14> A method for producing a genome-edited individual of a plant, the method comprising:
exposing a shoot apex of an axillary bud of a plant body;
introducing, into the shoot apex, a microparticle coated with at least one kind of nucleic acid and/or at least one kind of protein;
growing the shoot apex to obtain a plant body; and
selecting a genome-edited plant body from the plant body.

### INDUSTRIAL APPLICABLITY

The present invention can be used in, for example, agriculture, pharmaceutical industry, and enzyme industry.

1. A method for transforming a plant, the method comprising:
exposing a shoot apex of an axillary bud of a plant body; and
introducing, into the shoot apex, a microparticle coated with at least one nucleic acid.

2. The method according to claim 1, wherein the axillary bud of the plant body is an axillary bud of a plant body grown on a medium.

3. The method according to claim 1, wherein the axillary bud is an axillary bud of a lateral shoot.

4. The method according to claim 3, wherein the lateral shoot is a stolon.

5. The method according to claim 1, wherein the plant is any one plant selected from the group consisting of plants of the family Solanaceae.

6. The method according to claim 5, wherein the plants of the family Solanaceae are potatoes.

7. A method for producing a transformant of a plant, the method comprising:
exposing a shoot apex of an axillary bud of a plant body;
introducing, into the shoot apex, a microparticle coated with at least one nucleic acid;
growing the shoot apex so as to obtain a plant body; and
selecting a transformed plant body from the plant body.

8. A method for editing a plant genome, the method comprising:
exposing a shoot apex of an axillary bud of a plant body; and
introducing, into the shoot apex, a microparticle coated with at least one material selected from the group consisting of nucleic acid and protein.

9. The method according to claim 8, wherein the axillary bud of the plant body is an axillary bud of a plant body grown on a medium.

10. The method according to claim 8, wherein the axillary bud is an axillary bud of a lateral shoot.

11. The method according to claim 10, wherein the lateral shoot is a stolon.

12. The method according to claim 8, wherein the plant is any one plant selected from the group consisting of plants of the family Solanaceae.

13. The method according to claim 12, wherein the plants of the family Solanaceae are potatoes.

14. A method for producing a genome-edited individual of a plant, the method comprising:
exposing a shoot apex of an axillary bud of a plant body;
introducing, into the shoot apex, a microparticle coated with at least one material selected from the group consisting of nucleic acid and protein;
growing the shoot apex so as to obtain a plant body; and
selecting a genome-edited plant body from the plant body.


SEQUENCE LISTING (14 sequences)

SEQ ID NO: 1 - DNA, 23 bp, Artificial Sequence
  35S-F
ccagagggctattgagacttttc

SEQ ID NO: 2 - DNA, 23 bp, Artificial Sequence
  35S-R
atatagaggaagggtcttgcgaa

SEQ ID NO: 3 - RNA, 42 nt, Artificial Sequence
  crRNA GBSS1-tg1
agggcuguuaacaagcuugaguuuuagagcuaugcuguuuug

SEQ ID NO: 4 - RNA, 42 nt, Artificial Sequence
  crRNA GBSS1-tg2
gggcuguuaacaagcuugauguuuuagagcuaugcuguuuug

SEQ ID NO: 5 - RNA, 42 nt, Artificial Sequence
  crRNA GBSS1-tg3
uacuaagguaacacccaagaguuuuagagcuaugcuguuuug

SEQ ID NO: 6 - RNA, 42 nt, Artificial Sequence
  crRNA ALS
caagugccgaggaggaugauguuuuagagcuaugcuguuuug

SEQ ID NO: 7 - RNA, 69 nt, Artificial Sequence
  tracrRNA
aaacagcauagcaaguuaaaauaaggcuaguccguuaucaacuugaaaaaguggcaccgagucggugcu

SEQ ID NO: 8 - DNA, 24 bp, Artificial Sequence
  GBSS1 F
cttgcctactgtaatcggtgataa

SEQ ID NO: 9 - DNA, 23 bp, Artificial Sequence
  GBSS1 R
tttgacctgcagataaagtagcg

SEQ ID NO: 10 - DNA, 20 bp, Artificial Sequence
  ALS F
ggttccctggtgtttgcatt

SEQ ID NO: 11 - DNA, 20 bp, Artificial Sequence
  ALS R
gcttcacgaacaaccctagg

SEQ ID NO: 12 - RNA, 42 nt, Artificial Sequence
  crRNA-PDS-tg1
ggacucuugccagcaaugcuguuuuagagcuaugcuguuuug

SEQ ID NO: 13 - DNA, 20 bp, Artificial Sequence
  PDS F
tttccccgaagctttacccg

SEQ ID NO: 14 - DNA, 20 bp, Artificial Sequence
  PDS R
atctgtcaccctatccggca